(19) United States
(12) Patent Application Publication
(10) Pub. No.: US 2008/0221482 A1
(43) Pub. Date: Sep. 11, 2008

(54) STEERABLE TIP FOR FLEXIBLE RODS

(75) Inventors: Mark B. Mondry, Evergreen, CO (US); John E. Phillips, Arvada, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Labor Saving Devices, Inc., Commerce City, CO (US)

(21) Appl. No.: 11/682,819

(22) Filed: Mar. 6, 2007

(51) Int. Cl. A61M 25/01

(52) U.S. Cl. 600/585

(57) ABSTRACT

Various steerable tips may be used with flexible rods to move the tips through a wide assortment of cavities. In one embodiment, a steerable tip is constructed of a rigid tip body having a proximal end, a distal end and a plurality of radially extending fins. Also, at least one of the fins defines a surface through which extends at least one through hole. Also, a proximal connector is operably coupled to the proximal end of the tip body, and the proximal connector is adapted to couple the tip body to a rod. A distal connector is operably coupled to the distal end of the tip body. This permits other accessories to be coupled to the tip.

### BACKGROUND OF THE INVENTION

This invention relates generally to the field of accessory tips, particular for assisting rods in traversing cavities. More specifically, the invention relates to steerable tips for flexible rods.

Flexible rods, typically made from pulltruded fiberglass, have been used for retrofit wire and cable installation applications to push and pull wires and cables inside wall spaces, in crawl spaces, though overhead ceiling spaces and other structural spaces. These flexible rods have generally replaced the use of metal “fish tape” which historically has been used for similar purposes. Such rods typically have threaded connectors at each end to allow numerous rods to be connected together to provide long lengths of 30 feet or more. Various accessory tips have been developed that can be connected to such rods to add versatility, and to accommodate special installation and method requirements.

One existing tip incorporates a metal “whisk” type structure having four evenly spaced steel wire legs extending in a curved fashion. The legs are soldered together at the distal end to form a blunt end, and the rod may be coupled to the proximal end. An example of such a tip can be found in the current Labor Saving Devices Inc. product catalog (Volume 24) on numerous pages, including pages 35, 38, 40, 43 and 44. The complete disclosure of this catalog is herein incorporated by reference.

While such a tip has proven to be commercially acceptable, improvements are still desired. Hence, this invention relates to steerable tips with various novel features.

### BRIEF SUMMARY OF THE INVENTION

The invention provides various steerable tips for use with flexible rods. In one embodiment, a steerable tip comprises a rigid tip body having a proximal end, a distal end and a plurality of radially extending fins. Also, at least one of the fins includes at least one through hole. Also, a proximal connector is operably coupled to the proximal end of the tip body, and the proximal connector is adapted to couple the tip body to a rod. A distal connector is operably coupled to the distal end of the tip body. This permits other accessories or components to be coupled to the tip.

In some cases, one or more of the fins could include a slot which may be curved in geometry and include a sharpened edge. This slot may be useful in cutting into a wire or cable jacket to “capture” it from a distance, thus permitting the wire to be pulled toward the user. To remove the wire, it is simply pulled out of the slot.

In one particular arrangement, a pair of spaced apart through holes may be included in one or more of the surfaces. In this way, the wire may be threaded through both holes and then bent back and twisted onto itself to secure the wire to the tip. In some cases, one of the through holes may be elongate to allow the wire's jacket to lay close to the find to provide a low profile and reduce catching on other surfaces, such as ceiling tiles.

In one aspect, the tip body has a central axis extending between the proximal end and the distal end, and the fins extend radially outward from the central axis. In another aspect, the tip body includes three, four or more equally spaced apart fins.

In some cases, the tip body is constructed of a unitary piece of molded plastic, such as ABS. In other cases, the tip body may be constructed from aluminum alloys, biodegradable materials such as fiberboard, recyclable plastics, or composites, such as graphite, Kevlar and the like or various combinations thereof. In a further aspect, the tip body, or parts thereof, may be constructed of a luminescent material.

In a particular arrangement, each fin tapers outward from the proximal end toward the distal end, and is rounded at the distal end. In one particular arrangement, the fins have a length in the range from about two inches to about four inches and radially extend up to a distance in the range from about ½ inch to about 1½ inches.

According to one aspect, the proximal connector has a proximal end that may include a female end or a male threaded end. Also, the proximal connector may have a knurled distal end. Further, the knurled distal end may be molded into the tip body. Further, the distal connector may have a threaded end and a knurled end, and the knurled end may be molded into the tip body. However, the invention is not intended to be limited to such ends and may have other ends, such as threads that are internally fabricated within the tip body (at either the proximal end, the distal end, or both), other integrally formed connections, and the like

In a further embodiment, the invention provides a method for steering a rod. The method utilizes a steerable tip comprising a rigid tip body having a proximal end, a distal end and a plurality of radially extending fins. At least one of the fins defines a surface having at least one through hole extending through the surface, and a proximal connector is operably coupled to the proximal end of the tip body. Further, a distal connector is operably coupled to the distal end of the tip body. As part of the method, a rod is coupled to the proximal connector of the steerable tip. Also, the rod is grasped and manipulated to steer the rod through a cavity using the steerable tip.

In another step, a flexible cord is coupled to the tip through the through hole. Further, a tool may be coupled to the distal connector of the steerable tip.

In yet another embodiment, the invention provides a steerable tip system comprising a rigid tip body having a proximal end, a distal end and a plurality of radially extending fins. At least one of the fins includes at least one through hole. A proximal connector is operably coupled to the proximal end of the tip body. A distal connector is operably coupled to the distal end of the tip body. A flexible rod is also included in the kit and is configured to be coupled to the proximal connector. In some cases, the rod is constructed from fiberglass. In other cases, the rod may be constructed of other composite, plastic, nylon or other suitable flexible material.

### BRIEF DESCRIPTION OF THE DRAWINGS

FIG. 1 is a front view of one embodiment of a steerable tip according to the invention.

FIG. 2 illustrates the steerable tip of FIG. 1 rotated 90°.

FIG. 3 illustrates a proximal connector of the steerable tip of FIG. 2.

FIG. 4 is a front view of an alternative steerable tip with a different proximal connector according to the invention.

FIG. 5 illustrates the steerable tip of FIG. 4 rotated 90°.

FIG. 6 is a bottom view of the steerable tip of FIG. 4 with the proximal connector removed.

FIG. 7 illustrates the proximal connector of the steerable tip of FIG. 4.

FIG. 8 is a front view of still another alternative embodiment of a steerable tip according to the invention.

FIG. 9 illustrates the steerable tip of FIG. 8 rotated 45°.

FIG. 10 illustrates a top view of the steerable tip of FIG. 8.

FIG. 11 illustrates a distal connector of the steerable tip of FIG. 8.

FIG. 12 is an alternative embodiment of a steerable tip according to the invention.

FIG. 13 is a top view of the steerable tip of FIG. 12.

FIG. 14 illustrates the proximal end of the steerable tip of FIG. 12 rotated 90°.

FIG. 15 illustrates yet another embodiment of a steerable tip according to the invention.

FIG. 16 illustrates an embodiment of a steerable tip having a triangular fin according to the invention.

FIG. 17 illustrates an embodiment of a steerable tip having a round fin according to the invention.

FIG. 18 illustrates an embodiment of a steerable tip having a fin with a round tip and a flat edge according to the invention.

FIG. 19 illustrates an embodiment of a steerable tip having a fin with an angled tip and a flat edge according to the invention.

FIG. 20 illustrates an embodiment of a steerable tip having a paddle-shaped fin according to the invention.

FIG. 21 illustrates an embodiment of a steerable tip having an oval fin according to the invention.

FIG. 22 illustrates an embodiment of a steerable tip having a fin with a serrated edge according to the invention.

FIG. 23 illustrates another embodiment of a steerable tip according to the invention.

FIG. 24 is a perspective view of the tip of FIG. 23 showing a wire attached thereto according to the invention.

FIG. 25 is a cross sectional side view of the tip of FIG. 24.

### DETAILED DESCRIPTION OF THE INVENTION

The invention provides various steerable tips, methods for their use and wire and cable installation systems. The steerable tips are configured to be coupled to flexible rods and have various rigid fins to assist in moving the rod through various cavity configurations. The fins act as a series of supports when the rod is laying on its side to lift the distal end up and provide sufficient structure to allow the tip to longitudinally slide up and over small protrusions or irregularities on a surface. The fins also help to prevent the distal end of the rod from snagging or otherwise getting lodged in a corner, in insulation or in other materials.

In use, the rod is steered by rolling the rod (or rods if multiple rods are connected together), and therefore the tip, along its axis where the fins of the tip walk the distal end to either side depending upon the direction of rotation. The tip may also have a shape similar to a light bulb which allows the tip to travel across edges and irregularities longitudinally.

To provide such features, the tip is constructed of strong, yet lightweight material. Light weight is critical since the rods used for wire and cable installation are generally highly flexible to enable bending around angles of 90 degrees or greater and other contours. If the tip is too heavy, the weight may cause the rod to bend or sag, thereby compromising the necessary tactile positioning control needed by the installer/user to maneuver the rod to the desired position quickly and accurately.

Another feature is the use of one or more through holes in the fins. This permits a light weight nylon (or other material) “pull line” to be tied to the tip and pushed or pulled through a desired structure. This pull line is then used to actually pull the wire or cable being installed, thus avoiding loading the tip with the wire or cable itself. Alternatively, a wire or cable could be tied to the through hole directly.

The steerable tips of the invention may be constructed as a solid structure made from plastic or other stiff, durable, light weight material. The tips may be made of injection molded ABS plastic or other suitable materials and may have three or more fins. At least one of the fins defines a surface that may have one or more through holes or voids which extend through the surface to permit attachment of wire or cables to the tip by twisting or tying the stripped or jacketed wire through the hole and onto the tip. In one particular embodiment, the tip may be molded of a single piece and may incorporate four symmetrically spaced fins to provide a stable and balanced platform when lying on it side. However, other numbers of fins could be used as well. Multiple holes or other cut-outs can be provided in the fins to minimize weight providing the resultant fin structure provides adequate strength and streerability side to side. In addition, the fins surfaces of one or more of the fins provide an area to print, etch or cut out a logo, product part number or other identifier on the tip. This permits the ability to provide promotional identifiers, ownership identifiers or other materials to be placed onto the tip.

In one particular arrangement, the tip includes a connector on the proximal end to permit a tool or other attachment, such as a rod, sensor, magnet or accessory to be coupled to the tip. The proximal connector may be molded into, or as part of, the tip to provide increased strength and durability.

The plastic may be selected or formulated to incorporate a luminescent or glow-on-the-dark material to allow the tip to absorb light energy and emit the light in dark spaces, thereby allowing the user to easily see the location of the distal end of the rod in dark spaces, such as in crawl spaces, false ceilings, conduits, cable raceways, attics and the like.

The tips may also include a connector for connecting additional accessories and tips to the distal end of the tip. Such a connector may use a female threaded connector molded into the distal tip. Other mechanical connectors such as clevis pin, biased detent and capture window, friction fit, or other suitable mechanical connecting means could also be used. The distal connector permits additional accessories, attachments or tools, such as magnets, sensors, lights and various mechanical accessories, such as a j-shaped extension or other extension or embellishment to be attached to the distal tip to add functionality.

Although ABS plastic is one exemplary material to construct the tip, a variety of other materials may be used, such as lightweight 6061 aluminum, other suitable lightweight alloys, composites, such as carbon fibers, graphite, Kevlar or the like, or biodegradable materials such a fiber pulp resin matrix, fiberboard or the like. Alternatively, the steerable tips of the invention may incorporate a combination of materials assembled, affixed or molded together to form the desired solid structure. The materials may be ferrous or non-ferrous. Also, the connectors may be made as a friction fit or a snap together multi-component structure to allow easy disassembly and reduce space in storage.

In another aspect, the tips may include a hollow longitudinal channel to permit the rod to be inserted into the tip and be removably attached at the extreme distal end of the tip using a mechanical connector at that extreme distal end. In some cases, the channel permits the tip to function as a sleeve, i.e., the tip may be affixed or removably positioned at different points along the rod or may even float along the length of the rod to provide a movable attachment point for a wire.

A further aspect is that the tip may be covered with an airtight bladder that can be inflated to allow the tip to perform as a float when it is desired to position the rod across water or other fluid and avoid submerging. Another option is to place a foam material in between the fins to provide buoyancy. For example, the spaces between the fins may be filled or partially filled with a closed cell foam or other material to provide buoyancy. Also, this foam material may be luminescent.

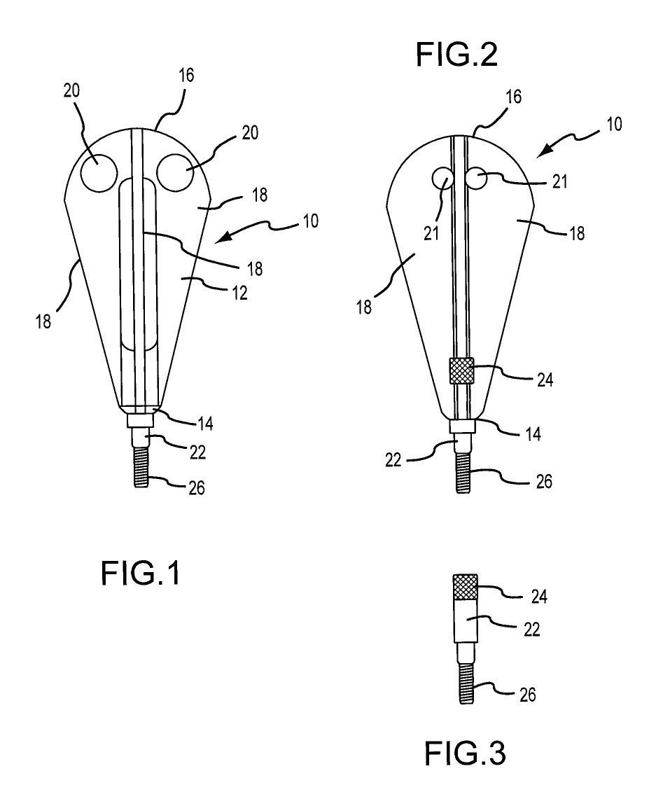

Referring now to FIG. 1, one embodiment of a steerable tip 10 will be described. Steerable tip 10 is constructed of a tip body 12 have a proximal end 14 and a distal end 16. Tip body 12 includes four radially extending fins 18 that are perpendicular to each other. Each of the fins defines various surfaces, which in some cases can be planar. For convenience and functionality, one or more through holes 20 and 21 may be disposed through fins 18 and permit various wires, cables, cords, strings, and the like to be coupled to tip 10.

Steerable tip 10 further includes a proximal connector 22 which is illustrated in greater detail in FIG. 3. Proximal connector 22 has a distal end 24 and a proximal end 26. Also, tip body 12 includes an opening in proximal end 14 into which distal end 24 of proximal connector 22 is inserted as best illustrated in FIG. 2. Preferably, distal end 22 is molded into proximal end 14 to form a secure connection between the two components. Alternatively this connection can be accomplished with adhesive or other suitable bonding material. Alternatively, the proximal end 14 of tip body 12 can be shaped to form the equivalent functionality of proximal connector 22 using suitable fabrication materials and techniques such as ABS plastic and injection molding techniques to reduce the number of manufactured components.

Distal end 26 of proximal connector 22 includes threads that permit proximal connector 22 to be coupled to one or more flexible rods. In this way, steerable tip 10 may easily be coupled to a flexible rod so that steerable tip 10 may be employed to help steer the rod when passing through a cavity. Also shown with a threaded end, it will be appreciated that proximal connector 22 may have a variety of coupling mechanisms, such as quick connects, snap fit connections, detent connections, friction fit connections, and the like. Some specific examples will be described hereinafter.

Tip body 12 may be constructed from essentially any suitable material that is light weight, durable and generally rigid. In some cases, tip body 12 could even be somewhat flexible. One particular material that may be used is an ABS plastic. However, a variety of other materials may be used, such as light weight metals, alloys, rigid or resilient composites, rubbers or plastics, and the like.

In some cases, fins 18 may have a length from proximal end 14 to distal end 16 that is in a range from about two inches to about four inches, and in some cases from about one inches to ten or more inches. Further, each fin 18 may radially extend out to about 0.5 inches to 1.5 inches, and in some cases from about one inches to five or more inches. The general angle of outward taper from proximal end 14 may be in the range from about 14 degrees to about 16 degrees, and in some cases from about 10 degrees to about 45 degrees. This taper may be made linear or nonlinear.

Through holes 20 and 21 may be provided through each fin 18, or only certain of the fins. In some cases, through holes 20 may have a circular diameter to facilitate ease of manufacture with a diameter in the range from about 0.125 inches to about 0.5 inches, and in some cases from about 0.0625 inches to about 0.5 inches or more. However, it will be appreciated that other shapes may be used, such as oblong, angular, geometric, or other various shapes providing the chosen shape comprises a void aperture of at lease 0.0625 inches in diameter. Also, while shown near distal end 16, it will be appreciated that through holes 20 and 21 may be provided at other locations within fin 18. Also, in some cases multiple through holes, of the same or varying shapes and sizes, may be provided in each fin 18.

One particular advantage of constructing tip body 12 of a light weight material is that it may be fabricated as a single body. For example, an injection molding process may be used to construct tip body 12 or a unified component having both tip body and proximal connector 22. In this way, steerable tip 10 may be manufactured in a relatively inexpensive manner, while still being light weight and durable.

In use, a flexible rod may be coupled to proximal connector 22 by screwing it to proximal end 26. Steerable tip 10 is then inserted into a structural space or cavity, and the rod is manipulated to move tip 10 through the cavity to a desired distal location. If needed, the rod may be rotated to permit steerable tip 10 to “walk” side-to-side across a surface through the cavity where the steerable tip, and therefore the distal end of the flexible rod attached to it, can be accurately positioned at a precise distal location around or behind structures. Also, before insertion into the cavity, a string, cord, wire, cable or the like may be coupled to tip body 12 through one or more of holes 20 so that it may be pulled through the cavity to physically connect a beginning or entry point to a desired end or exit point within a structure of cavity. When reaching the desired distal location, the string, cord, wire or cable may be uncoupled from tip 10 if in the desired final location, or may be tied, taped or otherwise secured to another usually heavier wire or cable so that it may be pulled through the cavity. In this way, new wire or cable can be installed in existing structure without the need to tear out wall surfaces or other structures in order to position the wire or cable through them. In another use, steerable tip 10 is used in a similar fashion to be able to retrieve a string, cord, wire or cable from a distant location. In this application, steerable tip 10 is used in conjunction with a flexible rod to position the distal end of steerable tip to a location within a structure or cavity where a string, cord, wire or cable can be retrieved and pulled back to the proximal end of the rod.

Another feature of steerable tip 10 is that distal end 16 is rounded or curved in order to facilitate movement of steerable tip 10 longitudinally over bumps, ridges, or the like. However, it will be appreciated that other shapes may be used at distal end 16. For example, distal end 16 could be more pointed, or in some cases the radius of curvature could be increased to flatten distal end 16 to provide more of a directional or pointed distal end 16 to enable distal end 16 to move through, or pierce, material such as insulating material or moisture barriers within structural walls and cavities, or to anchor the distal end 16 into such materials for later retrieval.

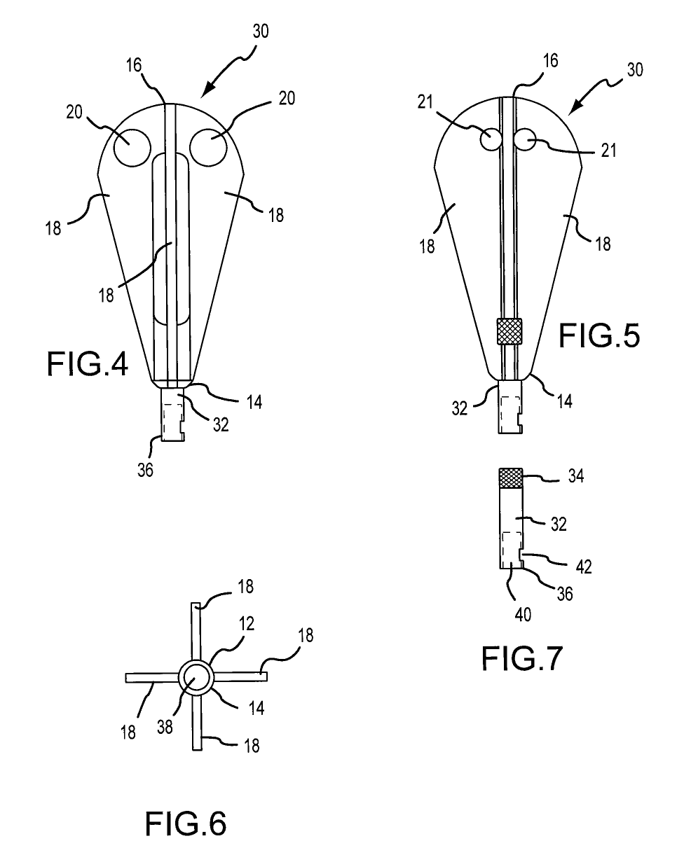

Referring now to FIGS. 4-7, another embodiment of a steerable tip 30 will be described. Steerable tip 30 may be constructed to be similar to steerable tip 10. As such, similar elements will be referred to using the same reference numerals used in connection with FIGS. 1-3. Also, it will be appreciated that the features of other embodiments discussed herein may also be used in connection with steerable tip 30. Proximal end 14 of tip body 12 includes a proximal connector 32 that is shown in greater detail in FIG. 7. Proximal connector 32 includes a proximal end 34 and a distal end 36. Proximal end 34 may be knurled or have other features similar to proximal connector 22 to permit it to be securely coupled to tip body 12. As best illustrated in FIG. 6, distal end 14 of tip body 12 includes a cavity 38 into which proximal end 34 of proximal connector 32 is inserted. Cavity 38 may have dimensions which are similar to the outer dimensions of proximal connector 32. For example, in some cases, the diameter of cavity 38 may be in the range from about 0.25 inches to about 0.375 inches, and in some cases from about 0.25 inches to about 2.00 inches. Tip body 12 may have outer dimensions at proximal end 14 that are in the range from about 0.30 inches to about 0.40 inches, and in some cases from about 0.30 inches to about 5.0 inches.

Proximal connector 32 may be constructed of a metal or other rigid material and includes an opening 40 and a through hole 42 which extends through a sidewall of proximal end 36. With such a configuration, proximal connector 32 may be coupled to a shaft which has a distal end that may fit within opening 40. Also, the distal end of the rod may include a detent, projection, or the like that is spring biased outwardly so as to snap within through hole 42 after passing beyond proximal end 36. To remove the rod, the detent may be depressed and then the rod may be pulled from proximal connector 32.

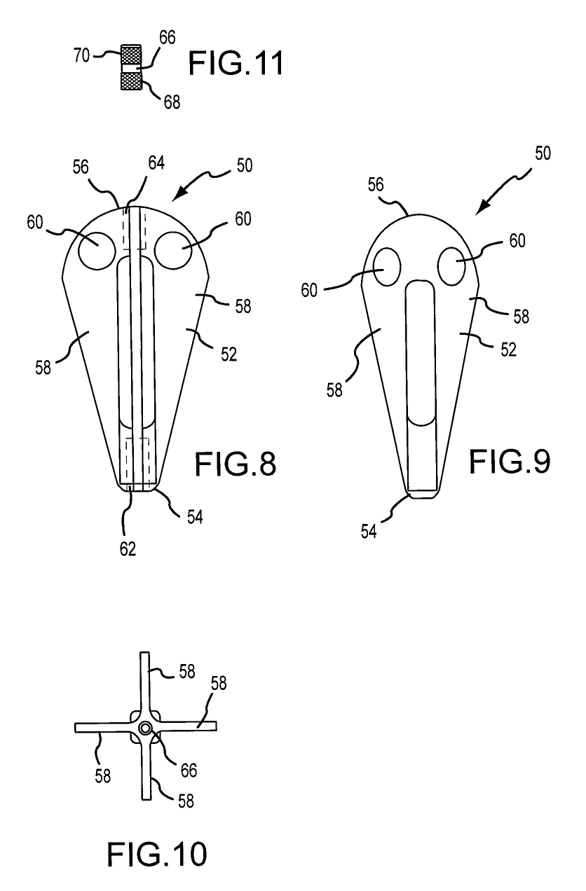

FIGS. 8-10 illustrate another embodiment of a steerable tip 50. Steerable tip 50 includes a tip body 52 that may be constructed of materials similar to those described in connection with other embodiments. Tip body 52 includes a proximal end 54 and a distal end 56. Tip body 52 also includes four fins 58 which each include a through hole 60. Fins 58 and through hole 60 may be constructed in a manner similar to those described with other embodiments. Tip body 52 may also include proximal opening 62 to permit a proximal connector to be coupled to tip body 52. The proximal connector may be any one of the proximal connectors described herein. Also, distal end 56 includes a distal opening 64 to permit a distal connector (see FIGS. 10 and 11) to be coupled to the distal end 56 of tip body 52. Distal connector 66 includes a proximal end 68 and a distal end 70. Distal connector 66 may be constructed of a metal or other rigid material and may include knurling or other features to permit it to be securely coupled with distal opening 64. For example, body 52 may be insert molded around distal connector 66, or may be bonded to connector 66. Alternatively, tip body 52 may be formed to have distal opening configured to include molded-in threads or other connecting structure to combine the structural functionality of distal connector 66 into tip body 52 without the need to have two separate components. Also, the proximal end may be configured in a similar manner. Distal connector 66 permits a wide variety of accessories and tips to be coupled to tip body 52. Distal connector 66 may include threads or other mechanical connector schemes to allow such accessories to be coupled to steerable tip 50. Merely by way of example, such additional accessories could include magnets, sensors, lights, j-shaped extensions, other extensions, or the like.

Although not shown, it will be appreciated that proximal opening 62 and distal opening 64 could be configured as an elongated channel extending entirely through tip body 52. In this way, tip body 52 would include a hollow longitudinal channel to allow a rod to be inserted completely through tip 50 to allow tip 50 to be positioned at different points along the rod.

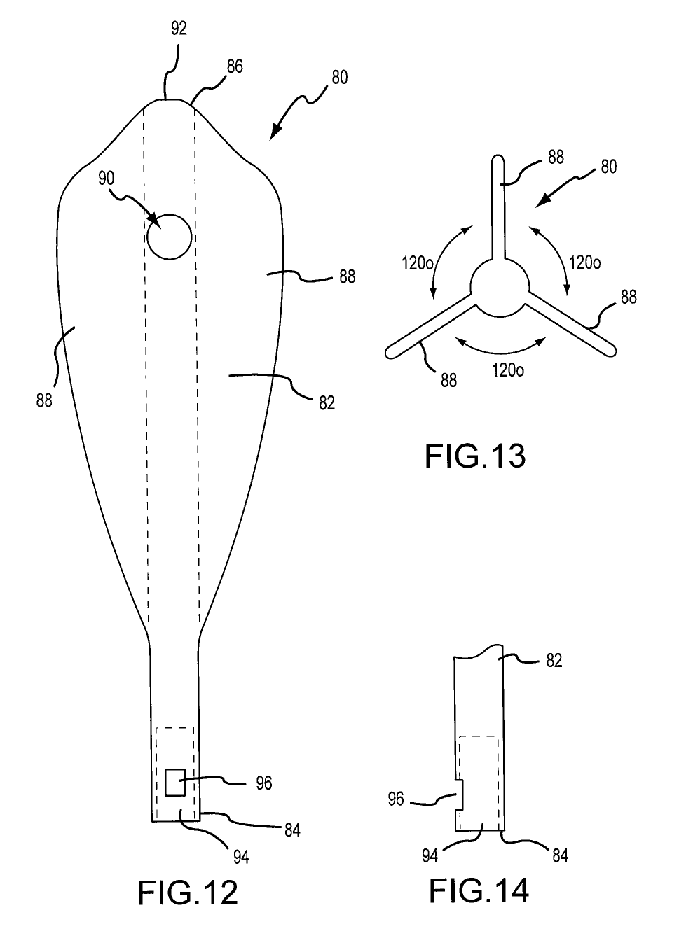

FIGS. 12-14 illustrate still another embodiment of a steerable tip 80. Steerable tip 80 is constructed of a tip body 82 that may be constructed of any of the materials described herein. Tip body 82 includes a proximal end 84, a distal end 86 and set of three fins 88. As shown in FIG. 13, each of the fins 88 is separated by 120 degrees. However, it will be appreciated that other numbers of fins may be provided. These fins may be equally spaced apart or in some cases unequally spaced apart. Tip body 82 defines a surface through which extends a through hole 90 for attaching a wire, cable, or the like. Also, it will be appreciated that through hole 90 could be included in one or more of fins 88.

Distal end 86 includes a blunt nose 92 which smoothly transforms into fins 88. Blunt nose 92 may be useful in helping tip 80 to traverse through tortuous cavities. Proximal end 84 further includes an opening 94 and a side wall through hole 96 similar to that described in connection with the embodiment illustrated in FIG. 4. In this way, a rod with a spring loaded detent may be easily snap fit to distal end 84.

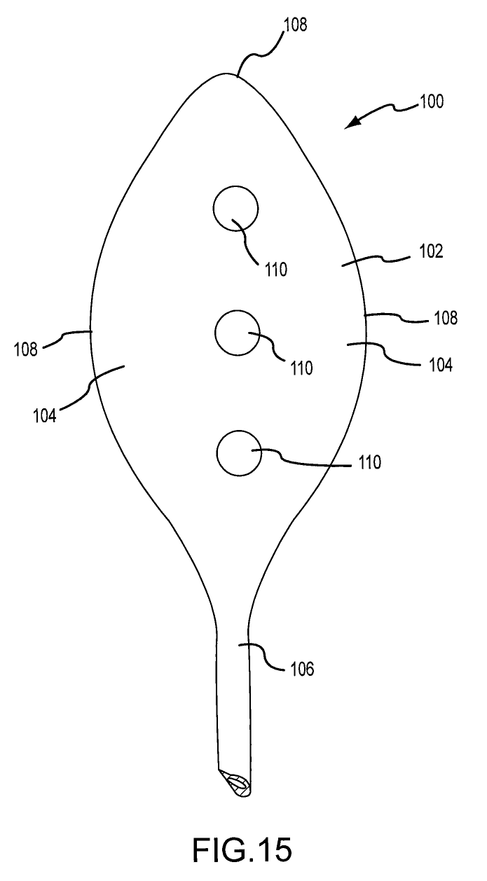

FIG. 15 illustrates yet another embodiment of a steerable tip 100 that is constructed of a tip body 102 that may be constructed in a manner similar to the other tip bodies described herein. One feature of tip body 102 is that it includes three or more fins 104 which provide tip 100 with a spoon shape. The fins 104 have a smooth ramp up from the proximal end 106 and then smoothly taper down to distal end 108. The blunt tip in combination with the smooth “ride up” plateau at distal end 108 permits tip 100 to be easily moved over ridges or other engaging surfaces. Also, fins 104 include a generally flat mid region 108 which is useful for stability and traction on other surfaces when steerable tip 100 is rotated along its longitudinal axis to steer steerable tip 100 from side to side. Also, through holes 110 of various shapes and sizes may be provided through the center of tip 100 or at various locations along fins 104 to permit wires or other cables to be coupled to tip 100.

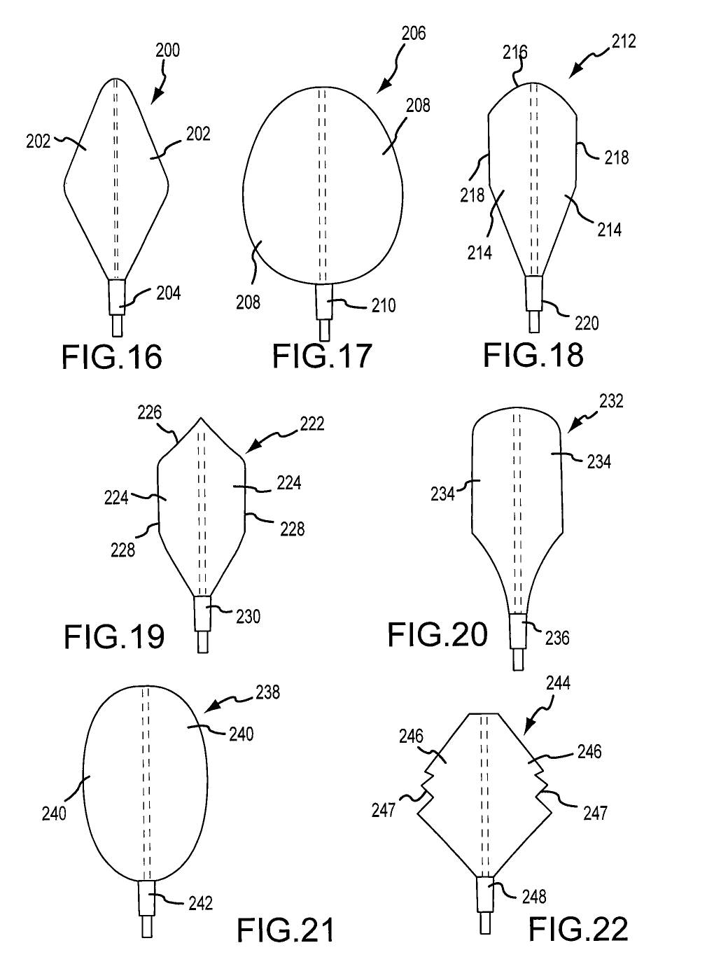

It will be appreciated that the steerable tips of the invention may take on a variety of other shapes. Some examples of such shapes are illustrated in FIGS. 16 through 22. Further, it will be appreciated that any of the tips in FIGS. 16 through 22 may be constructed using techniques similar to another of the other embodiments described herein and may include any of the features also set forth in those embodiments.

FIG. 16 illustrates a steerable tip 200 having four triangular fins 202. Also, tip 200 includes a proximal connector 204. FIG. 17 illustrates a steerable tip 206 having four round fins 208 and a proximal connector 210. FIG. 18 illustrates a steerable tip 212 having four fins 214 with a round tip 216 and a flat edge 218. Also, a proximal connector 220 is coupled to the proximal end. FIG. 19 illustrates a steerable tip 222 having fins 224 with an angled tip 226 and a flat edge 228. A proximal connector 230 is coupled to the proximal end. FIG. 20 illustrates a steerable tip 232 having paddle-shaped fins 234 and a proximal connector 236. FIG. 21 illustrates a steerable tip 238 having oval fins 240 and a proximal connector 242. FIG. 22 illustrates a steerable tip 244 having fins 246 with a serrated edge 247. A proximal connector 248 is coupled to the proximal end.

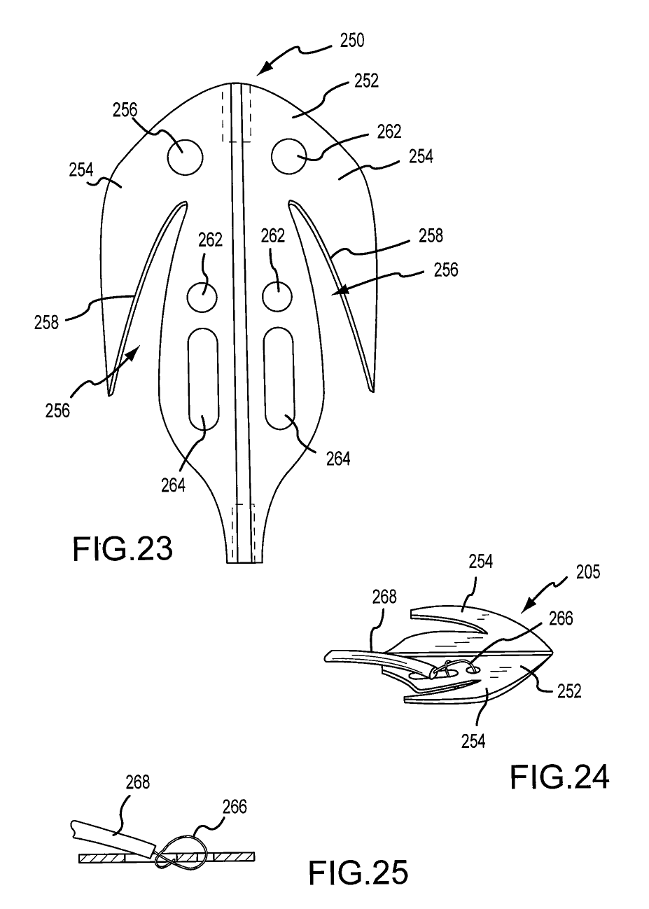

FIG. 23 illustrates another embodiment of a steerable tip 250 having a tip body 252 and a plurality of fins 254. Tip 250 may be similar to some respects to the other tips described herein and may include similar features, such as proximal connectors, distal connectors, numbers and sizes of fins, and the like. As such, these, and the various alternatives, will not be described in detail, recognizing that such components may be incorporated into tip 250.

One feature of tip 250 is that the fins 254 each include a slot 256 having a sharpened edge 258 on one side of the slot. Sharpened edges 258 act as a knife edge that cuts into the jacket surrounding the wire or cable. This permits a user to cut into and “capture” a wire from a distance, allowing the user to pull it towards him without losing the grip. To release the captured wire, it is simply pulled out of the slot 256.

Tip 250 inches a series of through holes which may be used in a manner similar to the through holes of other embodiments. For examples, through holes 260 may be included in each fin 254. Alternatively, or in addition to, one or more of the fins 254 may include a set of spaced apart through holes 262 and 264. In some cases, through holes 264 are elongate in geometry. Through holes 262 and 264 permit wire, cables and the like to be easily tied to tip 250. This is illustrated in greater detail in FIGS. 24 and 25. As shown, a wire cable 266 that is surrounded by a plastic jacket 268 is being coupled to tip 250. The end of cable 266 is bare (after removing jacket 268). Cable 266 is inserted through through hole 264 and then back up through through hole 262 where cable 266 is bent back and twisted onto itself to secure cable 266 to tip 250. The elongate geometry of through hole 264 permits jacket 268 to lay close to fin 254 to make a low profile and reduce catching on other surfaces.

Tip 250 may also include distal and proximal connectors at a distal end 270 and a proximal end 272 similar to other embodiments. Also, a lumen could extend between distal end 270 and proximal end 272 similar to other embodiments.

The invention have now been described in detail for purposes of clarity and understanding. However, it will be appreciated that certain changes and modifications may be practiced within the scope of the appended claims.

What is claimed is:

1. A steerable tip for use with a flexible rod, the tip comprising:
a generally rigid tip body having a proximal end, a distal end and a plurality of radially extending fins, wherein at least one of the fins defines a surface having at least one through hole extending through the surface;
a proximal connector operably coupled to the proximal end of the tip body, wherein the proximal connector is adapted to couple the tip body to a rod; and
a distal connector operably coupled to the distal end of the tip body.

2. A tip as in claim 1, wherein the tip body has a central axis extending between the proximal end and the distal end, and wherein the fins extend radially outward from the central axis, and wherein the fin surfaces are generally planar in geometry.

3. A tip as in claim 1, wherein the tip body includes at least three equally spaced apart fins.

4. A tip as in claim 1, wherein the tip body is constructed of a unitary piece of molded plastic.

5. A tip as in claim 1, wherein the tip body is constructed from a material selected from a group consisting of plastic, metal, and composites.

6. A tip as in claim 1, wherein the tip body is constructed having at least a portion thereof incorporating a luminescent material that can absorb and emit light energy.

7. A tip as in claim 1, wherein each fin tapers outward from the proximal end toward the distal end, and is rounded at the distal end.

8. A tip as in claim 1, wherein the proximal connector has a proximal end selected from a group of female ends and male ends, wherein the proximal connector has a knurled distal end, and wherein the knurled distal end is molded into the tip body.

9. A tip as in claim 1, wherein the proximal connector and the distal connector are integrally formed as part of the tip body.

10. A tip as in claim 1, wherein the distal connector has a threaded end and a knurled end, and wherein the knurled end is molded into the tip body.

11. A tip as in claim 1, wherein the distal connector comprises a snap-together, non-threaded feature to permit a snap-together fit with a tip element.

12. A tip as in claim 1, wherein the fins have a length in the range from about 2 inches to about 4 inches and radially extend up to a distance in the range from about 0.5 inches to about 1.5 inches.

13. A tip as in claim 1, wherein at least one of the fins includes a slot having a sharpened edge that is adapted to capture a wire.

14. A tip as in claim 1, wherein the surface includes a pair of spaced apart through holes, and wherein one of the through holes is larger than the other.

15. A method for steering a rod, the method comprising:
providing a steerable tip comprising a rigid tip body having a proximal end, a distal end and a plurality of radially extending fins, wherein at least one of the fins defines a surface having at least one through hole extending through the surface; a proximal connector operably coupled to the proximal end of the tip body; and a distal connector operably coupled to the distal end of the tip body
coupling a rod to the proximal connector of the steerable tip; and
grasping the rod and steering the rod through a cavity using the steerable tip.

16. A method as in claim 15, further comprising attaching a flexible cord to the tip through the through hole.

17. A method as in claim 16, further comprising manipulating the rod to position the cord in a desired location.

18. A method as in claim 15, further comprising coupling a tool to the distal connector of the steerable tip.

19. A steerable tip system comprising:
a rigid tip body having a proximal end, a distal end and a plurality of radially extending fins, wherein at least one of the fins defines a surface through which extends at least one through hole;
a proximal connector operably coupled to the proximal end of the tip body;
a distal connector operably coupled to the distal end of the tip body; and
a flexible rod that is configured to be coupled to the proximal connector.

20. A steerable tip system as in claim 19, wherein the rod is constructed from fiberglass.

21. A tip as in claim 19, wherein the tip body has a central axis extending between the proximal end and the distal end, and wherein the fins extend radially outward from the central axis.

22. A tip as in claim 19, wherein the tip body includes at least three generally equally spaced apart fins.

23. A tip as in claim 19, wherein the tip body is constructed of a unitary piece of molded plastic.

24. A steerable tip for use with a flexible rod, the tip comprising:
a rigid tip body having a proximal end, a distal end and at least three fins, wherein at least one of the fins includes at least one through hole, wherein the tip body has a central axis extending between the proximal end and the distal end, wherein the fins extend radially outward from the central axis, and wherein the tip body is constructed of a unitary piece of molded plastic;
a proximal connector operably coupled to the proximal end of the tip body, wherein the proximal connector is adapted to couple the tip body to a rod; and
a distal connector operably coupled to the distal end of the tip body.

25. A steerable tip for use with a flexible rod, the tip comprising:
a generally rigid tip body having a proximal end, a distal end and a plurality of radially extending fins, wherein at least one of the fins defines a surface having at least one through hole extending through the surface, and wherein at least one of the fins includes a slot having a sharpened edge that is adapted to capture a wire; and
a proximal connector operably coupled to the proximal end of the tip body, wherein the proximal connector is adapted to couple the tip body to a rod.

26. A tip as in claim 25, further comprising a distal connector operably coupled to the distal end of the tip body.
